(19) United States
(12) Patent Application Publication
(10) Pub. No.: US 2018/0228633 A1
(43) Pub. Date: Aug. 16, 2018

(54) LEG COMPRESSION SLEEVE WITH INSERT FOR SHIN SPLINTS

(71) Applicant: Michael J. Carey, Traverse City, MI (US)

(72) Inventor: Michael J. Carey, Traverse City, MI (US)

(21) Appl. No.: 15/897,517

(22) Filed: Feb. 15, 2018

Related U.S. Application Data
(60) Provisional application No. 62/459,216, filed on Feb. 15, 2017.

(51) Int. Cl. A61F 5/01; A61F 5/30

(52) U.S. Cl. CPC A61F 5/0104; A61F 5/30

(57) ABSTRACT

A compression sleeve for wearing over the calf/shin to relieve the discomfort of shin splints. The compression sleeve includes a pocket on its inner face at an upper end of the sleeve, the pocket sized to removably receive a large, flat, substantially incompressible pressure block insert with a substantially flat inner face configured to engage a significant portion of the tibialis anterior muscle on the upper, outer part of the calf with locally increased compressive force with respect to the remainder of the sleeve.

### RELATED APPLICATIONS/PRIORITY BENEFIT CLAIM

This application claims the benefit of U.S. Provisional Application No. 62/459,216, filed Feb. 15, 2017 by the same inventor (Carey), the entirety of which provisional application is hereby incorporated by reference.

### FIELD

The subject matter of the present application is in the field of leg compression sleeves or socks.

### BACKGROUND

Compression sleeves for the lower leg, using enhanced features for locally focused compression, are known.

U.S. Patent Application Publication US 2003/0069531 A1 (Hall) shows a multilayer compression sleeve with a discontinuous inner layer of slip-resistant microdots applied in a pattern to impart enhanced compression to certain areas of a human leg knee joint.

U.S. Patent Application Publication US 2016/0000612 A1 (Cox) shows a therapeutic pressure band with elongate rubber tube pressure members to apply discrete areas or points of pressure in a line across a muscle, tendon, bone, or muscle group generally perpendicular to a longitudinal axis of a lower leg to treat shin splints.

U.S. Pat. No. 8,801,645 (Lipshaw et al.) shows a compression garment wrapped around a lower leg, with a plurality of bands attachable to the wrap at different locations to wrap around the limb and the wrap and to apply compression force.

Prior attempts to solve the problem of shin splint pain using compression sleeves or wraps seem to rely on large-area fabric compression, or small highly-detailed compression features that would seem to be of marginal effectiveness or require undue positioning skill, or soft compressible members combined with the elastic fabric of the sleeve that might not provide consistent relief.

### BRIEF SUMMARY

The present invention is a compression sleeve for the lower leg, i.e. the calf/shin portion below the knee, which reduces the pain from shin splints while running.

The compression sleeve comprises an elongated sleeve of elastic fabric configured to wrap around the calf/shin with fairly uniform compressive force. An upper outer portion of the sleeve corresponding to the tibialis anterior muscle on the lateral anterior (upper, outer) side of the shin is provided with an inside pocket accessible from the upper edge of the sleeve, and is sized to removably receive a large, solid, substantially incompressible, flat-faced pressure “block”.

The pressure block applies a relatively large flat uniform area of additional localized pressure or compression to the tibialis anterior muscle. This large-area pressure is believed to engage the (S)T-37 acupuncture point located on the middle of the tibialis anterior muscle, without requiring specialized acupuncture knowledge or training, and with a large margin of error for lay users.

“Block” is used herein to mean a relatively large insert with at least one substantially flat interior face having at least a couple of square inches of surface area sufficient to cover a middle portion of the tibialis anterior muscle, and being made from a material that is incompressible relative to the anterior tibialis muscle and the force applied by the compression sleeve and sufficiently thick and rigid for locally increased compressive force with respect to the remainder of the sleeve. While a rectangular and/or trapezoidal shape is preferred, the block is not limited to these shapes. The block may be comprised of measurements of, for example, width: 2-3 cm, depth or thickness: 1-2 cm, length: 5-15 cm.

The compression sleeve fabric may optionally be a split sleeve, with vertical sides or edges secured to one another for a desired level of compression.

These and other features and advantages of the invention will become apparent from the detailed description below, in light of the accompanying drawings.

### BRIEF DESCRIPTION OF THE DRAWINGS

FIG. 1 is a perspective view of a person's lower leg with a compression sleeve according to one example of the invention, and showing the pressure block insert exploded from the compression sleeve.

FIG. 2 is similar to FIG. 1, but shows the compression sleeve being applied to a wearer's leg.

FIG. 3 is a perspective view of the pressure block insert of FIG. 1.

FIG. 3A is an edge view of the pressure block insert of FIG. 1.

FIGS. 4A and 4B are perspective views of compression sleeves according to FIG. 1 removed from the leg, the sleeve in FIG. 4A being a continuous tubular sleeve and the sleeve of FIG. 4B being a split sleeve fastenable at the edges.

FIG. 5 is a plan view of the inner side of the compression sleeve of FIG. 1 laid flat, with seam and/or fastener lines shown in broken lines.

### DETAILED DESCRIPTION

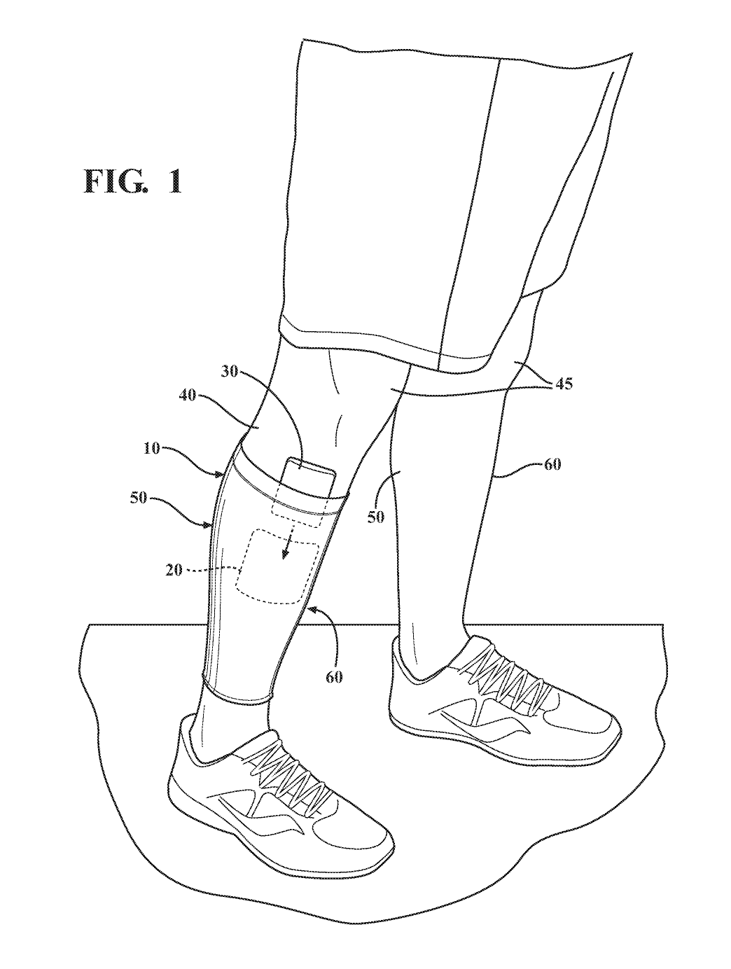

FIG. 1 shows a compression sleeve 10 in exemplary form in order to teach how to make and use the claimed invention. Compression sleeve 10 is formed from a strong elastic fabric of known type designed to fit tightly with a compressive fit around a person's leg 40 below the knee 45, i.e. around the calf 50 and shin 60. The size of the sleeve, the elasticity of the fabric in the sleeve, and other conventional features of the sleeve fabric may vary in known manner to fit a wide range of leg sizes and to offer different levels of compression.

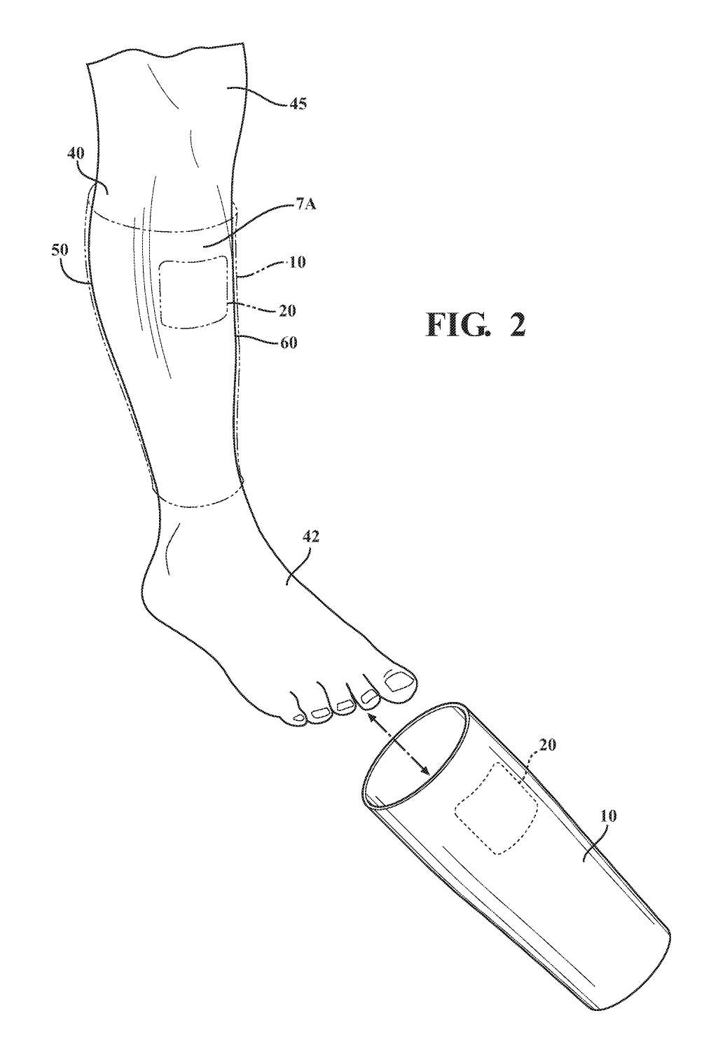

In accordance with the invention, compression sleeve 10 includes an interior pocket 20 formed on the inside surface of an upper, outer side or panel of the sleeve, “outer” being defined by the respective leg on which the sleeve is worn and off-center from the shin corresponding to the tibialis anterior muscle 7A (FIG. 2), “upper” meaning in the top half or so of the sleeve, depending on the sleeve height. Pocket 20 includes a removable pressure block insert 30, in the illustrated example a smooth-surfaced, rounded rectangular block of plastic, hard foam, wood, or other substantially incompressible material having a substantially smooth, flat inner face designed to be pressed against the leg by the elastic sleeve 10 when inserted in pocket 20.

Because the length and shape of individual calves vary, the width and length of pocket 20 may have a width and length greater than the width and length of pressure block insert 30 in order to adjust the position of the block in sleeve 10 so that the block is better centered over the tibialis anterior muscle until optimal pain relief is achieved. Multiple pressure block inserts 30 of different size may also be supplied with a sleeve 10 to allow an individual to adjust pressure and fit with respect to the tibialis anterior muscle, and pocket 20 may be sized accordingly to accommodate a range of block sizes.

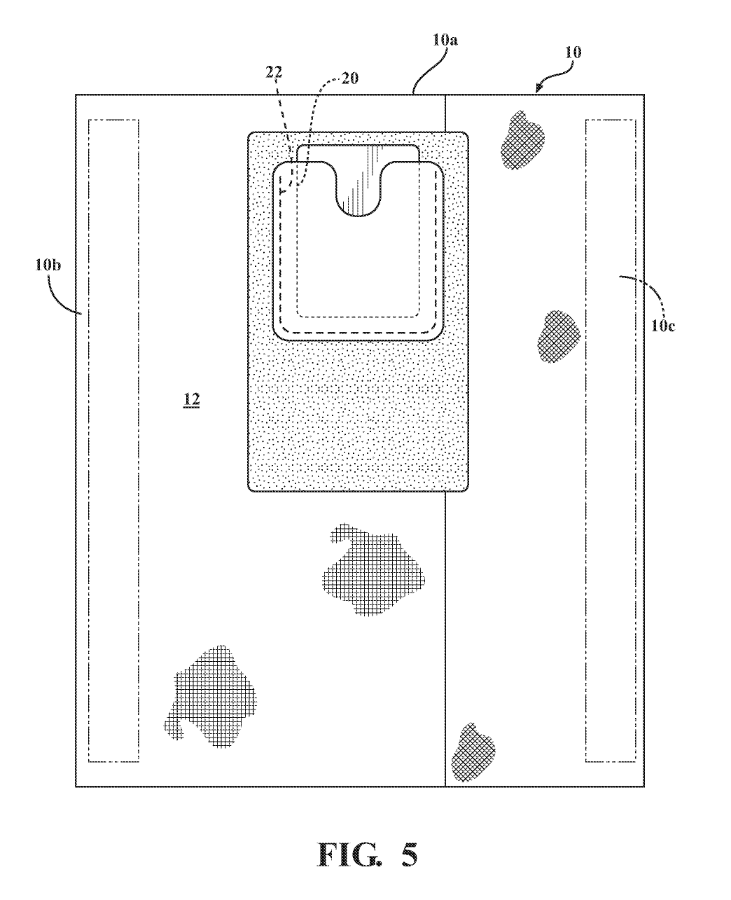

As best shown in FIGS. 4B and 5, pocket 20 may be formed by sewing suitable pocket fabric to the inside face 12 of sleeve 10, the sewing shown schematically by broken lines 22, leaving the upper edge of the pocket open for inserting the pressure block 30. The pocket fabric is preferably elastic in order to match and stretch with the fabric of the sleeve. Alternately, pocket 20 may be removably secured to the inside face of sleeve 10, for example with a border of Velcro® hook-and-loop or similar fastener secured to the inside perimeter of the sides and bottom of pocket 20 to removably grip the inner fabric face of the sleeve, broken lines 22 also serving to represent such a detachable border on pocket 20. Removability would allow pocket 20 to be re-positioned in sleeve 10 to better position pressure block insert 30 over the tibialis anterior muscle, particularly if the pocket is sized just large enough to receive the block. The inside face 12 of sleeve 10 in the upper outer part of the sleeve may be faced with a soft fuzzy loop-type material 12a that mates with a male hook-portion of the Velcro® fastener border 22 on the inside face of the sides and lower edge of pocket 20.

While pocket 20 is preferably formed from an elastic fabric similar to that used for sleeve 10, the pocket may alternately be formed from a non-elastic fabric or fabric equivalent that is resistant to folding in order to make inserting the pressure block 30 easier, provided the pocket material is sized to accommodate the stretching of the outer sleeve elastic (for an integral pocket), or where the pocket is removably attached to the inside of the sleeve.

FIG. 2 shows pressure block 30 partially exploded from the pocket 20 and the sleeve 10, representing either the removal or insertion of block 30 in the pocket. Pocket 20 opens upwardly toward top edge 10a of the sleeve, with its upper open edge adjacent the top edge of the sleeve so that block 30 can be inserted and removed from the pocket by sliding the block in a vertical direction underneath the elastic fabric of sleeve top edge 10a when the sleeve is being worn.

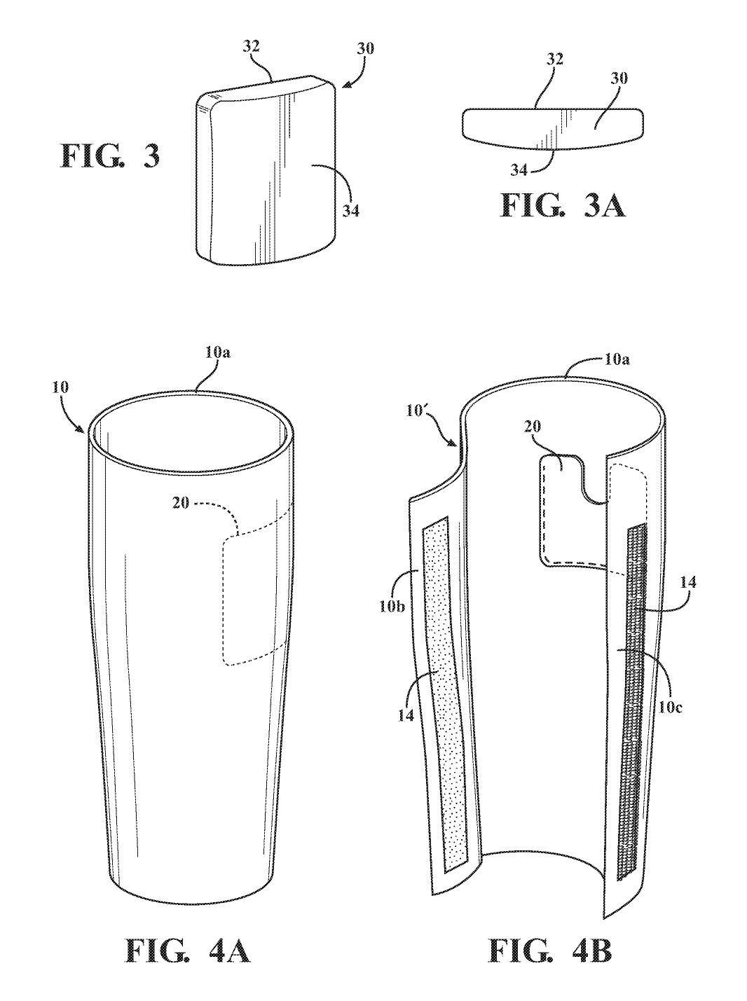

Referring to FIGS. 2, 3 and 3A, block 30 has a substantially flat inner face 32 that applies a substantially uniform pressure to the tibialis anterior muscle when inserted in pocket 20. The surface area of inner face 32 on block 30 is sufficiently large to engage a substantial portion of the middle area of the tibialis anterior muscle 7A, the block surface area preferably being for example on the order of two square inches or greater. The surface area of inner face 32 is accordingly greater than the surface area of calf 50 corresponding to the (S)T-37 acupuncture point in the region of the tibialis anterior, making it easy to engage the key (S)T-37 acupuncture point in that muscle without the need to precisely locate a particular feature of the block relative to a precise point on the leg.

The outer face 34 of pressure block 30 may be curved in convex fashion to present a smoother outer surface more similar to the outer contours of the shin/calf, and for more even pressure distribution from the elastic sleeve over the face of the pressure block. The corners and edges of pressure block 30 are preferably rounded.

FIGS. 1 and 2 show sleeve 10 while being worn, the broken lines representing pocket 20 underneath the sleeve fabric also illustrating the location of block 30 inserted in the pocket over the tibialis anterior muscle 7A.

FIGS. 2 and 4A show a continuous tubular sleeve 10 configured to be put on or taken off lengthwise over the foot 42. FIG. 4B shows an alternate construction of tubular sleeve 10′ wherein the sleeve is split lengthwise to form two securable rear edges 11, 13 that can be fastened together to compress the leg, for example, with Velcro® fastener 14. Split sleeve 10′ can be applied laterally to leg 40 and secured around the calf/shin, rather than longitudinally as shown in FIG. 2, which might be preferable for some individuals.

FIG. 5 represents sleeve 10 or 10′ laid flat, for example as a pattern before being sewn together along seams to form a continuous tubular sleeve, or when fasteners along edges 11, 13 are separated to open the sleeve. In the illustrated example a larger panel corresponding to the front of the leg and containing pocket 20 is separated from a smaller panel corresponding to the back of the leg by a flat-felled seam 11, the multi-panel construction allowing the option of using different fabrics of differing elasticity for the two panel portions. While FIG. 5 represents a currently preferred seam or fastenable edge configuration for sleeve 10, it will be understood that other sewing, knitting, or fastening configurations should be possible.

It will finally be understood that the disclosed embodiments represent presently preferred examples of how to make and use the invention, but are intended to enable rather than limit the invention. Variations and modifications of the illustrated examples in the foregoing written specification and drawings may be possible without departing from the scope of the invention. It should further be understood that to the extent the term “invention” is used in the written specification, it is not to be construed as a limiting term as to number of claimed or disclosed inventions or discoveries or the scope of any such invention or discovery, but as a term which has long been conveniently and widely used to describe new and useful improvements in science and the useful arts. The scope of the invention supported by the above disclosure should accordingly be construed within the scope of what it teaches and suggests to those skilled in the art, and within the scope of any claims that the above disclosure supports in this application or in any other application claiming priority to this application.

1. A compression sleeve configured to fit around the calf and shin of a human wearer, the sleeve comprising:
an elongated sleeve at least a portion of which comprises elastic fabric, the sleeve configured to wrap around the calf/shin with substantially uniform compressive force;
an upper outer portion of the sleeve corresponding to the tibialis anterior muscle on the lateral anterior side of the shin comprising an inside pocket accessible from and having an upper opening adjacent an upper edge of the sleeve;
a pressure block insert of substantially incompressible material configured to slide into and out of the upper opening of the pocket, the pressure block insert having an inner face positioned in the pocket facing inwardly toward a wearer's leg, the inner face having an area sufficient to cover a substantial portion of the tibialis anterior muscle on a wearer's calf in compressive contact therewith when inserted in the pocket.

2. The compression sleeve of claim 1, wherein the inner face of the pressure block insert is substantially flat.

3. The compression sleeve of claim 1, wherein the elongated sleeve comprises a split sleeve with vertical side edges removably secured to one another.

4. The compression sleeve of claim 1, wherein the pressure block insert comprises a convexly curved outer face.

5. A method for reducing pain from shin splints, comprising:
applying a compression sleeve around the calf and shin of a human wearer, the compression sleeve comprising an elongated sleeve at least a portion of which comprises elastic fabric, the sleeve wrapping around the calf and shin with substantially uniform compressive force;
inserting a pressure block insert of substantially incompressible material into an upper opening of an inside pocket on an upper outer portion of the compression sleeve corresponding to the tibialis anterior muscle on the lateral anterior side of the shin, the inside pocket accessible from and comprising an upper opening adjacent an upper edge of the sleeve, and the pressure block insert comprising an inner face positioned in the pocket facing inwardly toward the wearer's leg, the inner face having an area sufficient to cover a substantial portion of the tibialis anterior muscle on the calf and held in compressive contact therewith by the compression sleeve.

6. The method of claim 5, wherein the inner face of the pressure block insert is substantially flat and applies substantially uniform pressure to the tibialis anterior muscle over an area corresponding to the inner face.
